(19) United States
(12) Patent Application Publication
(10) Pub. No.: US 2008/0188543 A1
(43) Pub. Date: Aug. 7, 2008

(54) PHARMACEUTICAL COMPOSITIONS CONTAINING SUBSTITUTED INDOLE ACID DERIVATIVES AS INHIBITORS OF PLASMINOGEN ACTIVATOR INHIBITOR-1 (PAI-1)

(75) Inventors: Arun A. THAKUR, Hants (GB); Mannching Sherry Ku, Thiells, NY (US); Hsueh-Ling Wu, Edison, NJ (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 12/026,268

(22) Filed: Feb. 5, 2008

Related U.S. Application Data
(60) Provisional application No. 60/899,575, filed on Feb. 5, 2007.

(51) Int. Cl. A61K 31/405; A61P 25/28; A61P 9/10; A61P 9/14; A61P 7/02

(52) U.S. Cl. 514/419

(57) ABSTRACT

Pharmaceutical compositions containing compounds of formula I are provided:

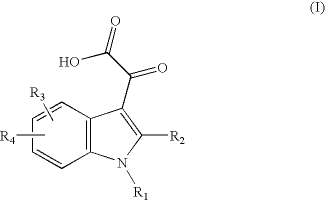

wherein the constituent variables are as defined herein. The compounds are inhibitors of plasminogen activator inhibitor-1 (PAI-1) and the compositions are useful for treating conditions resulting from fibrinolytic disorders, such as deep vein thrombosis and coronary heart disease, Alzheimer's disease and pulmonary fibrosis.

### CROSS-REFERENCE TO RELATED APPLICATION

This application claims priority benefit of U.S. Provisional Application Ser. No. 60/899,575 filed Feb. 5, 2007, which is incorporated herein by reference in its entirety.

### FIELD OF THE INVENTION

This invention relates to pharmaceutical formulations containing substituted indole acid derivatives as inhibitors of plasminogen activator inhibitor-1 (PAI-1) useful for the treatment of a wide variety of conditions including deep vein thrombosis, coronary heart disease, pulmonary fibrosis, cognition impairment, senility and Alzheimer's disease.

### BACKGROUND

Plasminogen activator inhibitor-1 (PAI-1) is a major regulatory component of the plasminogen-plasmin system. PAI-1 is the principal physiologic inhibitor of both tissue type plasminogen activator (tPA) and urokinase type plasminogen activator (uPA). Elevated plasma levels of PAI-1 have been associated with thrombotic events as indicated by animal experiments (Krishnamurti, Blood, 69, 798 (1987); Reilly, Arteriosclerosis and Thrombosis, 11, 1276 (1991); Carmeliet, Journal of Clinical Investigations, 92, 2756 (1993)) and clinical studies (Rocha, Fibrinolysis, 8, 294, 1994; Aznar, Haemostasis 24, 243 (1994)). Antibody neutralization of PAI-1 activity resulted in promotion of endogenous thrombolysis and reperfusion (Biemond, Circulation, 91, 1175 (1995); Levi, Circulation 85, 305, (1992)). Elevated levels of PAI-1 have also been implicated in diseases of women such as polycystic ovary syndrome (Nordt, Journal of Clinical Endocrinology and Metabolism, 85, 4, 1563 (2000)) and bone loss induced by estrogen deficiency (Daci, Journal of Bone and Mineral Research, 15, 8, 1510 (2000)). Accordingly, agents that inhibit PAI-1 would be of utility in treating conditions originating from fibrinolytic disorder such as deep vein thrombosis, coronary heart disease, pulmonary fibrosis, polycystic ovary syndrome, etc.

PAI-1 inhibitors, by virtue of their ability to lead to the activation of plasmin, are predicted to reduce the levels of both soluble and aggregated forms of Aβ40/42 peptide by enhanced proteolytic clearance. Since Aβ40/42 comprise amyloid plaques associated with Alzheimer's disease, use of the formulations of this invention are promising candidates for the prevention/treatment of Alzheimer's disease.

The present invention describes pharmaceutical formulations containing certain indole-containing, PAI-1 inhibitors for use in treating various conditions where PAI-1 inhibition is desirable.

### SUMMARY

This invention relates to pharmaceutical compositions containing compounds of formula (I), or a pharmaceutically acceptable salt, solvate or ester thereof:

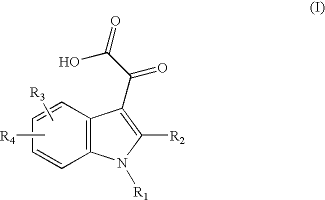

wherein:
R₁ is selected from C₁-C₈ alkyl, (—CH₂)ₙ—C₃-C₆ cycloalkyl, wherein n is an integer of from 0 to 3, pyridinyl, —CH₂-pyridinyl, phenyl or benzyl, the rings of the cycloalkyl, pyridinyl, phenyl and benzyl groups being optionally substituted by, from 1 to 3 groups selected from, halogen, C₁-C₄ alkyl, C₁-C₃ perfluoroalkyl, —O—C₁-C₃ perfluoroalkyl, C₁-C₃ alkoxy, —OH, —NH₂, or —NO₂;
R₂ is selected from H, C₁-C₆ alkyl, C₃-C₆ cycloalkyl, —CH₂—C₃-C₆ cycloalkyl, or C₁-C₃ perfluoroalkyl, —CH₂OH or CH₂OAc;
R₃ is selected from H, halogen, C₁-C₆ alkyl, C₁-C₃ perfluoroalkyl, C₁-C₆ alkoxy, C₃-C₆ cycloalkyl, —CH₂—C₃-C₆ cycloalkyl, C₄-C₆ cycloalkenyl, —CH₂—C₄-C₆ cycloalkenyl, —NH₂, or —NO₂;
R₄ is phenyl substituted by from 1 to 3 groups selected from halogen, C₁-C₄ alkyl, C₁-C₃ perfluoroalkyl, preferably —CF₃, —O—C₁-C₃ perfluoroalkyl, preferably —O—CF₃, C₁-C₃ alkoxy, —OH, —NH₂, —NO₂ or (CO)C₁-C₆ alkyl; and
one or more surfactants.

In some embodiments of this invention, said composition comprises from about 1% to 25% of said one or more surfactants.

In some embodiments of this invention, said compound of formula (I) is a compound of formula (II) or formula (III), or a pharmaceutically acceptable salt, solvate or ester thereof:

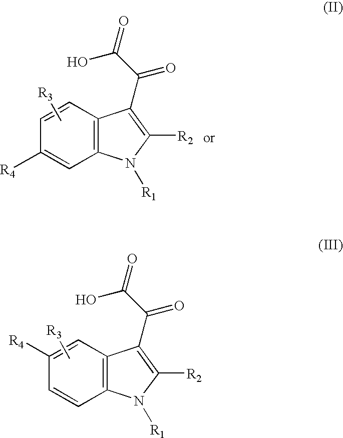

wherein:
R₁, R₂, R₃, and R₄ are as defined for previously in formula (I).

In some embodiments of this invention, the compound of formula (I) is a compound of formula (IV) or formula (V), or a pharmaceutically acceptable salt, solvate or ester thereof:

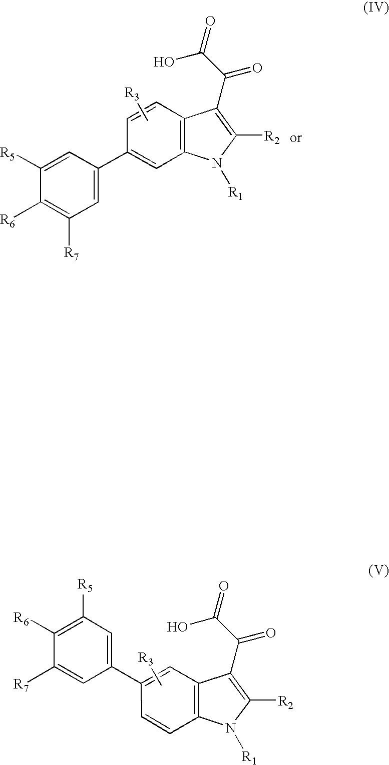

wherein:
R₁ is selected from C₁-C₈ alkyl, C₃-C₆ cycloalkyl, —CH₂—C₃-C₆ cycloalkyl, or benzyl, the rings of the cycloalkyl and benzyl groups being optionally substituted by from 1 to 3 groups selected from halogen, C₁-C₄ alkyl, C₁-C₃ perfluoroalkyl, —O—C₁-C₃ perfluoroalkyl, C₁-C₃ alkoxy, —OH, —NH₂, or —NO₂;
R₂ is selected from H, C₁-C₆ alkyl, C₃-C₆ cycloalkyl, —CH₂—C₃-C₆ cycloalkyl, or C₁-C₃ perfluoroalkyl;
R₃ is selected from H, halogen, C₁-C₆ alkyl, C₁-C₃ perfluoroalkyl, C₁-C₆ alkoxy, C₃-C₆ cycloalkyl, —CH₂—C₃-C₆ cycloalkyl, —NH₂, or —NO₂; and
R₅, R₆ and R₇ are independently selected from H, halogen, C₁-C₃ alkyl, C₁-C₃ perfluoroalkyl, —O—C₁-C₃ perfluoroalkyl, C₁-C₃ alkoxy, —OH, —NH₂, or —NO₂, provided that at least one of R₅, R₆ and R₇ is not H.

In some embodiments of this invention, the compound of formula (I) is a compound of formula (VI), or a pharmaceutically acceptable salt, solvate or ester thereof:

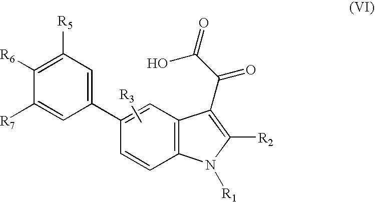

wherein:
R₁ is selected from benzyl, the benzyl group being optionally substituted by from 1 to 3 groups selected from halogen, C₁-C₄ alkyl, C₁-C₃ perfluoroalkyl, —O—C₁-C₃ perfluoroalkyl, or C₁-C₃ alkoxy;
R₂ is H;
R₃ is H; and
   R₅, R₆ and R₇ are independently selected from H, halogen, C₁-C₃ alkyl, C₁-C₃ perfluoroalkyl, —O—C₁-C₃ perfluoroalkyl and C₁-C₃ alkoxy, provided that at least one of R₅, R₆ and R₇ is not H.

In some embodiments of this invention, the compound of formula (I) is
{1-Methyl-6-[4-(trifluoromethoxy)phenyl]-1H-indol-3-yl}(oxo)acetic acid;
{1-Methyl-6-[4-(trifluoromethyl)phenyl]-1H-indol-3-yl}(oxo)acetic acid;
{1-Ethyl-6-[4-(trifluoromethoxy)phenyl]-1H-indol-3-yl}(oxo)acetic acid;
{1-Ethyl-6-[4-(trifluoromethyl)phenyl]-1H-indol-3-yl}(oxo)acetic acid;
{1-Benzyl-6-[4-(trifluoromethoxy)phenyl]-1H-indol-3-yl}(oxo)acetic acid;
{1-Benzyl-6-[4-(trifluoromethyl)phenyl]-1H-indol-3-yl}(oxo)acetic acid;
{1-[4-(tert-Butyl)benzyl]-6-[4-(trifluoromethyl)phenyl]-1H-indol-3-yl}(oxo)acetic acid;
{1-[4-(tert-Butyl)benzyl]-6-[4-(trifluoromethoxy)phenyl]-1H-indol-3-yl}(oxo)acetic acid;
{1-Benzyl-5-[4-(trifluoromethyl)phenyl]-1H-indol-3-yl}(oxo)acetic acid;
{6-[4-(tert-Butyl)phenyl]-1-methyl-1H-indol-3-yl}(oxo)acetic acid;
[5-(4-Acetylphenyl)-1-benzyl-1H-indol-3-yl](oxo)acetic acid;
{1-Benzyl-5-[4-(trifluoromethoxy)phenyl]-1H-indol-3-yl}(oxo)acetic acid;
{1-Benzyl-4-[4-(trifluoromethyl)phenyl]-1H-indol-3-yl}(oxo)acetic acid;
{1-Benzyl-5-[4-(tert-butyl)phenyl]-1H-indol-3-yl}(oxo)acetic acid;
[1-Benzyl-5-(3-chloro-4-fluorophenyl)-1H-indol-3-yl](oxo)acetic acid;
{1-Benzyl-5-[3,5-bis(trifluoromethyl)phenyl]-1H-indol-3-yl}(oxo)acetic acid;
{1-Benzyl-7-[4-(trifluoromethoxy)phenyl]-1H-indol-3-yl}(oxo)acetic acid;
[1-Benzyl-7-(3-chloro-4-fluorophenyl)-1H-indol-3-yl](oxo)acetic acid;
{1-(4-tert-Butylbenzyl)-5-[4-(trifluoromethoxy)phenyl]-1H-indol-3-yl}(oxo)acetic acid;
{1-Benzyl-4-[4-(trifluoromethoxy)phenyl]-1H-indol-3-yl}(oxo)acetic acid;
[1-Benzyl-6-(3-chlorophenyl)-1H-indol-3-yl](oxo)acetic acid;
{1-Benzyl-5-[3-(trifluoromethoxy)phenyl]-1H-indol-3-yl}(oxo)acetic acid;
{1-(4-Methylbenzyl)-5-[4-(trifluoromethoxy)phenyl]-1H-indol-3-yl}(oxo)acetic acid;
{1-(4-Fluorobenzyl)-5-[4-(trifluoromethoxy)phenyl]-1H-indol-3-yl}(oxo)acetic acid;
[1-Butyl-5-(4-chlorophenyl)-1H-indol-3-yl](oxo)acetic acid;
[1-Butyl-5-(3-chlorophenyl)-1H-indol-3-yl](oxo)acetic acid;
[1-Butyl-5-(3-methoxyphenyl)-1H-indol-3-yl](oxo)acetic acid;
[1-Butyl-5-(4-methoxyphenyl)-1H-indol-3-yl](oxo)acetic acid;
{1-Butyl-5-[4-(trifluoromethyl)phenyl]-1H-indol-3-yl}(oxo)acetic acid;
[1-(4-tert-Butylbenzyl)-5-(3-methylphenyl)-1H-indol-3-yl](oxo)acetic acid;
[1-(4-tert-Butylbenzyl)-5-(3-methoxyphenyl)-1H-indol-3-yl](oxo)acetic acid;
[1-(4-tert-Butylbenzyl)-5-(4-tert-butylphenyl)-1H-indol-3-yl](oxo)acetic acid;
[1-(4-tert-Butylbenzyl)-5-(3-chlorophenyl)-1H-indol-3-yl](oxo)acetic acid;
[1-(4-tert-Butylbenzyl)-5-(4-chlorophenyl)-1H-indol-3-yl](oxo)acetic acid;
[1-(4-tert-Butylbenzyl)-5-(2-methylphenyl)-1H-indol-3-yl](oxo)acetic acid;
{1-(2-Ethylbutyl)-5-[4-(trifluoromethoxy)phenyl]-1H-indol-3-yl}(oxo)acetic acid;
{2-[(Acetyloxy)methyl]-1-(4-methylbenzyl)-5-[4-(trifluoromethoxy)phenyl]-1H-indol-3-yl}(oxo)acetic acid;
{2-(Hydroxymethyl)-1-(4-methylbenzyl)-5-[4-(trifluoromethoxy)phenyl]-1H-indol-3-yl}(oxo)acetic acid;
{2-[(Acetyloxy)methyl]-1-benzyl-5-[4-(trifluoromethoxy)phenyl]-1H-indol-3-yl}(oxo)acetic acid;
{1-Benzyl-2-(hydroxymethyl)-5-[4-(trifluoromethoxy)phenyl]-1H-indol-3-yl}(oxo)acetic acid;
[5-(3-Chlorophenyl)-1-cyclopentyl-1H-indol-3-yl]-oxo-acetic acid;
[5-(3-chlorophenyl)-1-(cyclobutylmethyl)-1H-indol-3-yl](oxo)acetic acid;
[5-(3-chlorophenyl)-1-(3-methylcyclopropyl)-1H-indol-3-yl](oxo)acetic acid;
[5-(3-chlorophenyl)-1-(cyclohexylmethyl)-1H-indol-3-yl](oxo)acetic acid;
5-(4-trifluoromethylphenyl)-1-(cyclopentyl)-1H-indol-3-yl](oxo)acetic acid;
[5-(4-trifluoromethylphenyl)-1-(cyclobutylmethyl)-1H-indol-3-yl](oxo)acetic acid;
[5-(4-trifluoromethylphenyl)-1-(3-methylcyclopentyl)-1H-indol-3-yl](oxo)acetic acid;
[5-(4-trifluoromethylphenyl)-1-(cyclohexylmethyl)-1H-indol-3-yl](oxo)acetic acid;
[5-(4-trifluoromethylphenyl)-1-(cyclopentylpropyl)-1H-indol-3-yl](oxo)acetic acid;
[5-(3-trifluoromethylphenyl)-1-(cyclopentyl)-1H-indol-3-yl](oxo)acetic acid;
[5-(3-trifluoromethylphenyl)-1-(cyclobutylmethyl)-1H-indol-3-yl](oxo)acetic acid;
[5-(3-trifluoromethylphenyl)-1-(3-methylcyclopentyl)-1H-indol-3-yl](oxo)acetic acid;
[5-(3-trifluoromethylphenyl)-1-(cyclohexylmethyl)-1H-indol-3-yl](oxo)acetic acid;
[5-(3-trifluoromethylphenyl)-1-(cyclopentylpropyl)-1H-indol-3-yl](oxo)acetic acid;
[5-(4-methoxyphenyl)-1-(cyclohexylmethyl)-1H-indol-3-yl](oxo)acetic acid;
   or a pharmaceutically acceptable salt, solvate or ester thereof.

### BRIEF DESCRIPTION FOR THE DRAWINGS

FIG. 1. Depicts a powder X-ray diffraction pattern of [1-(4-tert-butylbenzyl)-5-(3-methylphenyl)-1H-indol-3-yl](oxo)acetic acid polymorph A, where the diffraction angle (20) scan ranges from 5 to 300.

FIG. 2. Depicts a differential scanning calorimetry (DSC) trace of [1-(4-tert-butylbenzyl)-5-(3-methylphenyl)-1H-indol-3-yl](oxo)acetic acid polymorph A, using a scan range 37 up to 200° C., scan rate 10° C./min.

FIG. 3 Depicts the pH-solubility profile of [1-(4-tert-butylbenzyl)-5-(3-methylphenyl)-1H-indol-3-yl](oxo)acetic acid.

FIG. 4 Depicts the effect of solubilizing agents on [1-(4-tert-butylbenzyl)-5-(3-methylphenyl)-1H-indol-3-yl](oxo)acetic acid at pH 4.8.

FIG. 5. Depicts release of [1-(4-tert-butylbenzyl)-5-(3-methylphenyl)-1H-indol-3-yl](oxo)acetic acid from wet granulation formulations containing representative surfactants used in this invention.

### DETAILED DESCRIPTION

This invention provides novel formulations containing indole-based PAI-1 inhibitors of formula I. One of skill in the art can appreciate the difficulties inherent in providing formulations for compounds that are lipophilic and acidic. Such compounds, due to the presence of a polar portion(s) together with a hydrophobic portion can present numerous difficulties to the task of providing a formulation that is capable of providing significant levels of the active moiety into the subject's bloodstream. One of the difficulties is in providing a formulation that will protect the compound from decomposition while simultaneously helping to solubilize the drug for purposes of enhancing absorption. Clearly, the need to solubilize the drug must be weighed against not introducing excess excipients which might exacerbate loading and stability problems. The present invention describes highly useful, novel and effective formulations for the delivery of compounds of formula I.

In one embodiment, compositions of this invention comprise a compound of formula (I) in a range of about 1% to 50% w/w of the composition. In another embodiment, the composition of this invention comprises a compound of formula (I) in a range of about 3.33% to 33.33% w/w of the composition. In some embodiments, the composition of this invention comprises a compound of formula (I) in about 50% w/w of the composition. In some embodiments, the composition of this invention comprises a compound of formula (I) in about 45% w/w of the composition. In some embodiments, the composition of this invention comprises a compound of formula (I) in about 40% w/w of the composition. In some embodiments, the composition of this invention comprises a compound of formula (I) in about 35% w/w of the composition. In some embodiments, the composition of this invention comprises a compound of formula (I) in about 33.33% w/w of the composition. In some embodiments, the composition of this invention comprises a compound of formula (I) in about 30% w/w of the composition. In some embodiments, the composition of this invention comprises a compound of formula (I) in about 25% w/w of the composition. In some embodiments, the composition of this invention comprises a compound of formula (I) in about 20% w/w of the composition. In some embodiments, the composition of this invention comprises a compound of formula (I) in about 15% w/w of the composition. In some embodiments, the composition of this invention comprises a compound of formula (I) in about 10% w/w of the composition. In some embodiments, the composition of this invention comprises a compound of formula (I) in about 5% w/w of the composition. In certain embodiments, the composition of this invention comprises a compound of formula (I) in about 3.33% w/w of the composition.

In some embodiments of this invention, a surfactant or combination of surfactants is employed. Surfactants are commonly known in the art and non-limiting definitions and examples can be found in, for example, Remington's Pharmaceutical Sciences, (21^{st} edition, Mack Publishing Company), which is herein incorporated by reference in its entirety. In certain embodiments, one or more of the surfactants employed in the invention is an anionic surfactant. In some embodiments, one or more of the surfactants employed in this invention is cationic. In certain embodiments of this invention, one or more of the surfactants used is zwitterionic. In some embodiments, one or more of the surfactants used may be neutral. While in no way wishing to be bound by examples given, some of the surfactants useful in the compositions of this invention include sodium docusate, cyclodextrins (e.g. 2-Hydroxypropyl-δ-cyclodextrin), polysorbates (e.g. polysorbate 80), centrimide, TPGS (d-alpha-tocopheryl polyethylene glycol 1000 succinate), sodium lauryl sulfate, and poloxamers. In some embodiments, the composition of this invention comprises a surfactant, wherein said surfactant is TPGS. In some embodiments, the composition of this invention comprises a surfactant, wherein said surfactant is sodium lauryl sulfate. In some embodiments, the composition of this invention comprises a combination of surfactants including sodium lauryl sulfate and TPGS. The surfactant or surfactants employed may be used in a range of between about 1% to 25% w/w of the composition. In some embodiments, the surfactant or surfactants employed may be present in a range of from about 5% to 20% w/w of the composition. In some embodiments, the surfactant or surfactants employed maybe present in a range of from about 5% to 15% w/w of the composition. In some embodiments, the surfactant or surfactants employed may be present in a range of from about 7% to 12.5% w/w of the composition. In some embodiments, the surfactant or surfactants employed may be present in about 25% w/w of the composition. In some embodiments, the surfactant or surfactants employed may be present in about 20% w/w of the composition. In some embodiments, the surfactant or surfactants employed may be present in about 15% w/w of the composition. In some embodiments, the surfactant or surfactants employed may be present in about 10% w/w of the composition. In some embodiments, the surfactant or surfactants employed may be present in about 5% w/w of the composition.

In some embodiments of this invention, a filler or combination of fillers (sometimes referred to as diluents) is employed in the compositions of this invention. The function of fillers as pharmaceutical excipients is well known to those of skill in the art and non-limiting definitions and examples can be found in, for example, Remington's Pharmaceutical Sciences, (21^{st} edition, Mack Publishing Company), which is herein incorporated by reference in its entirety. While in no way wishing to be bound by the examples given, some useful fillers that maybe used singly or in combination include, for example, mannitol, starch, maltose, lactose, sucrose, fructose, calcium phosphate, calcium carbonate, calcium sulphate, cellulose, dextrose, sorbitol, cellulose acetate, sodium alginate, maltodextrin, simethicone, and polydextrose. The filler or fillers as used in the compositions of this invention may be present in varying amounts and in some instances, an excipient may function as more than a filler. For example, a compound that performs the function of a binder may also function be categorized as a filler in certain cases. For purposes of the instant application, where the composition does not generically or specifically identify a binder, than the amount of binder used will be combined with the filler. So, for example, if a formulation identifies a compound and a filler, but not a binder, the percentage of the composition that is attributed to the filler will be also understood to possibly include a binder. Conversely, where the composition does generically or specifically list a binder, than the filler percentage will pertain to a filler (or fillers) itself. In certain embodiments of this invention, the filler is present in a range of from about 10% to 85% w/w of the composition. In some embodiments of this invention, the filler is present in a range of from 15% to 80% w/w of the composition. In some embodiments of this invention, the filler is present in a range of from about 20% to 75% w/w of the composition. In some embodiments of this invention, the filler is present in a range of from about 25% to 75% w/w of the composition. In some embodiments of this invention, the filler is present in a range of from about 30% to 75% w/w of the composition. In some embodiments of this invention, the filler is present in a range of from about 35% to 75% w/w of the composition. In some embodiments of this invention, the filler is present in a range of from about 40% to 75% w/w of the composition. In some embodiments of this invention, the filler is present in a range of from about 45% to 75% w/w of the composition. In some embodiments of this invention, the filler is present in a range of from about 50% to 75% w/w of the composition. In some embodiments of this invention, the filler is present in a range of from about 50% to 80% w/w of the composition.

In some embodiments of this invention, a binder or combination of binders is employed in the compositions of this invention. The function of binders as pharmaceutical excipients is well known to those of skill in the art and non-limiting definitions and examples can be found in, for example, Remington's Pharmaceutical Sciences, (21^{st} edition, Mack Publishing Company), which is herein incorporated by reference in its entirety. While in no way wishing to be bound by the examples given, some useful binders that maybe used singly or in combination include, for example, povidone, carbomers, polyoxamers, starch, methyl cellulose, chitosan, sodium alginate, sucrose, maltose, and gelatin. In certain embodiments, the binder is present in from about 1% to 35% w/w of the composition. In certain embodiments, the binder is present in from about 3% to 30% w/w of the composition. In certain embodiments, the binder is present in from about 5% to 25% w/w of the composition. In certain embodiments, the binder is present in from about 10% to 20% w/w of the composition. In some embodiments, the binder is present in about 15% w/w of the composition.

In some embodiments of this invention, a disintegrant or combination of disintegrants is employed in the compositions of this invention. The function of disintegrants as pharmaceutical excipients is well known to those of skill in the art and non-limiting definitions and examples can be found in, for example, Remington's Pharmaceutical Sciences, (21^{st} edition, Mack Publishing Company), which is herein incorporated by reference in its entirety. While in no way wishing to be bound by the examples given, some useful disintegrants that maybe included in the compositions of this invention are, for example, croscarmellose sodium, sodium starch glycolate, povidone and starch. In some embodiments, the disintegrant is present in from about 1% to 15% w/w of the composition. In some embodiments, the disintegrant is present in from about 2% to 12% w/w of the composition. In some embodiments, the disintegrant is present in from about 2% to 10% w/w of the composition. In some embodiments, the disintegrant is present in from about 2% to 8% w/w of the composition. In some embodiments, the disintegrant is present in about 5% w/w of the composition.

In some embodiments of this invention, a glidant or combination of glidants is/are employed in the compositions of this invention. The function of glidants as pharmaceutical excipients is well known to those of skill in the art and non-limiting definitions and examples can be found in, for example, Remington's Pharmaceutical Sciences, (21^{st} edition, Mack Publishing Company), which is herein incorporated by reference in its entirety. While in no way wishing to be bound by the examples given, some useful glidants that maybe included in the compositions of this invention are, for example, colloidal silicon dioxide, talc, magnesium silicate, and calcium silicate. In some embodiments, the glidant is present in from about 0.01% to 5% w/w of the composition. In some embodiments, the glidant is present in from about 0.1% to 3% w/w of the composition. In some embodiments, the glidant is present in from about 0.1% to 2% w/w of the composition. In some embodiments, the glidant is present in from about 0.1% to 1% w/w of the composition. In some embodiments, the glidant is present in from about 0.5% w/w of the composition. In some embodiments, the glidant is present in about 0.5% w/w of the composition.

In some embodiments of this invention, a lubricant or combination of lubricants is employed in the compositions of this invention. The function of lubricants as pharmaceutical excipients is well known to those of skill in the art and non-limiting definitions and examples can be found in, for example, Remington's Pharmaceutical Sciences, (21^{st} edition, Mack Publishing Company), which is herein incorporated by reference in its entirety. While in no way wishing to be bound by the examples given, some useful lubricants that maybe included in the compositions of this invention are, for example, magnesium stearate, sodium stearyl fumarate and stearic acid. In some embodiments, the lubricant is present in from about 0.01% to 5% w/w of the composition. In some embodiments, the lubricant is present in from about 0.1% to 3% w/w of the composition. In some embodiments, the lubricant is present in from about 0.1% to 2% w/w of the composition. In some embodiments, the lubricant is present in from about 0.1% to 1% w/w of the composition. In some embodiments, the lubricant is present in about 0.5% w/w of the composition.

The various structural embodiments of this invention as described by the various formulae presented, may be formulated according to the procedures described in this application.

The preferred salt forms of the compounds herein include but are not limited to sodium salts and potassium salts. Other useful salt forms of these compounds include those formed with pharmaceutically acceptable inorganic and organic bases known in the art. Salt forms prepared using inorganic bases include hydroxides, carbonates or bicarbonates of the therapeutically acceptable alkali metals or alkaline earth methals, such as sodium potassium, magnesium, calcium and the like. Acceptable organic bases include amines, such as benzylzmine, mono-, di- and trialkylamines, preferably those having alkyl groups of from 1 to 6 carbon atoms, more preferably 1 to 3 carbon atoms, such as methylamine, dimethylamine, trimethylamine, ethylamine, diethylamine, triethylamine, mono-, di-, and triethanolamine. Also useful are alkylene diamines containing up to 6 carbon atoms, such as hexamethylenediamine; cyclic saturated or unsaturated bases containing up to 6 carbon atoms, including pyrrolidine, peperidine, morpholine, piperazine and their N-alkyl and N-hydroxyalkyl derivatives, such as N-methyl-morpholine and N-(2-hyroxyethyl)-piperidine, or pyridine. Quaternary salts may also be formed, such as tetralkyl forms, such as tetramethyl forms, alkyl-alkanol forms, such as methyl-triethanol or trimethyl-monoethanol forms, and cyclic ammonium salt forms, such as N-methylpyridinium, N-methyl-N-(2-hydroxyethyl)-morpholinium, N,N-di-methylmorpholinium, N-methyl-N-(2-hydroxyethyl)-morpholinium, or N,N-dimethyl-piperidinium salt forms. These salt forms may be prepared using the acidic compound(s) of Formula I and procedures known in the art.

Ester forms of the compounds of this invention include straight chain alkyl esters having from 1 to 6 carbon atoms or branched chain alkyl groups containing 3 or 6 carbon atoms, including methyl, ethyl, propyl, butyl, 2-methylpropyl and 1,1-dimethylethyl esters. Other esters useful with this invention include those of the formula —COOR₇ wherein R₇ is selected from the formulae:

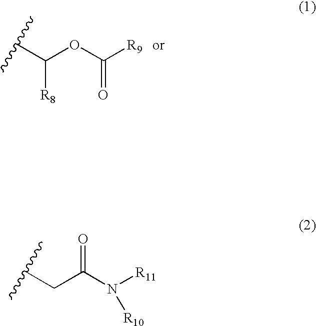

wherein R₈, R₉, R₁₀, R₁₁ are independently selected from hydrogen, alkyl of from 1 to 10 carbon atoms, aryl of 6 to 12 carbon atoms, arylalkyl of from 6 to 12 carbon atoms; heteroaryl or alkylheteroaryl wherein the heteroaryl ring is bound by an alkyl chain of from 1 to 6 carbon atoms.

Among the preferred ester forms of the compounds herein include but not limited to C₁-C₆ alkyl esters, C₃-C₆ branched alkyl esters, benzyl esters, etc. As used herein, the terms alkyl, alkenyl and alkynyl include both straight chain as well as branched claim chains. Preferably, the C₁-C₃ perfluoroalkyl substituent is —CF₃; the —O—C₁-C₃ perfluoroalkyl substituent is OCF₃; and the —S—C—C₃ perfluoroalkyl substituent is —SCF₃. At various places in the present specification, substituents of compounds of the invention are disclosed in groups or in ranges. It is specifically intended that the invention include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

As used herein, “aryl” refers to an unsaturated aromatic carbocyclic group of from 6 to 14 carbon atoms having a single ring (e.g., phenyl) or multiple condensed (fused) rings (e.g., naphthyl or anthryl). Preferred aryl groups include phenyl, naphthyl and the like. As used herein, ‘heteroaryl’, as defined herein, whether alone or as part of another group, refers to a mono- or bicyclic aromatic ring system containing 5-10 ring members of which 1-5 ring members are heteroatoms selected from N, O or S. At least one of the rings of the bicyclic ring system is heteroaromatic. Such heteroaryl groups can have a single ring, such as pyridyl, pyrrolyl or furyl groups, or multiple condensed rings, such as indolyl, indolizinyl, benzofuranyl or benzothienyl groups. Preferred heteroaryls include pyridyl, pyrrolyl and furyl.

Unless otherwise limited by the definition for the aryl or heteroaryl groups herein, such groups can optionally be substituted with from 1 to 5 substituents selected from the group consisting of acyloxy, hydroxy, acyl, alkyl of 1 to 6 carbon atoms, alkoxy of 1 to 6 carbon atoms, alkenyl of 2 to 6 carbon atoms, alkynyl of 2 to 6 carbon atoms, substituted alkyl, substituted alkoxy, substituted alkenyl, substituted alkynyl, amino, amino substituted by one or two alkyl groups of from 1 to 6 carbon atoms, aminoacyl, acylamino, azido, cyano, halo, nitro, thioalkoxy of from 1 to 6 carbon atoms, substituted thioalkoxy of from 1 to 6 carbon atoms, and trihalomethyl. Substituents on the alkyl, alkenyl, alkynyl, thioalkoxy and alkoxy groups mentioned above include halogens, CN, OH, and amino groups. Preferred substituents on the aryl groups herein include alkyl, alkoxy, halo, cyano, nitro, trihalomethyl, and thioalkoxy.

The compounds in this invention may contain one or more asymmetric centers, which can thus give rise to optical isomers (enantiomers) and diastereomers. While shown without respect to the stereochemistry in Formula I, the present invention includes such optical isomers (enantiomers) and diastereomers (geometric isomers); as well as the racemic and resolved, enantiomerically pure R and S stereoisomers; as well as other mixtures of the R and S stereoisomers and pharmaceutically acceptable salts thereof. The use of these compounds is intended to cover the racemic mixture or either of the chiral enantiomers.

Optical isomers can be obtained in pure form by standard procedures known to those skilled in the art, and include, but are not limited to, diastereomeric salt formation, kinetic resolution, and asymmetric synthesis. See, for example, Jacques, et al., Enantiomers, Racemates and Resolutions (Wiley Interscience, New York, 1981); Wilen, S. H., et al., Tetrahedron 33:2725 (1977); Eliel, E. L. Stereochemistry of Carbon Compounds (McGraw-Hill, NY, 1962); Wilen, S. H. Tables of Resolving Agents and Optical Resolutions p. 268 (E. L. Eliel, Ed., Univ. of Notre Dame Press, Notre Dame, IN 1972), each of which is incorporated herein by reference in their entireties.

The compositions of the present invention are inhibitors of the serine protease inhibitor PAI-1, and are therefore useful in the treatment, inhibition, prevention or prophylaxis in a mammal, preferably in a human, of those processes which involve the production and/or action of PAI-1 (a “PAI-1-associated disorder”). Thus, the compositions of the invention are useful in the treatment or prevention of noninsulin dependent diabetes mellitus and cardiovascular, ocular or kidney disease caused by such condition, and prevention of thrombotic events associated with coronary artery and cerebrovascular disease. These compositions are also useful for inhibiting the disease process involving the thrombotic and prothrombotic states which include, but are not limited to, formation of atherosclerotic plaques, venous and arterial thrombosis, myocardial ischemia, atrial fibrillation, deep vein thrombosis, coagulation syndromes, pulmonary fibrosis, cerebral thrombosis, thromboembolic complications of surgery (such as joint replacement), and peripheral arterial occlusion. These compositions are also useful in treating ischemic events such as stroke, associated with or resulting from atrial fibrillation.

The compositions of the invention may also be used in the treatment of diseases associated with extracellular matrix accumulation, including, but not limited to, renal fibrosis, chronic obstructive pulmonary disease, polycystic ovary syndrome, restenosis, renovascular disease and organ transplant rejection.

The compositions of the invention may also be used in the treatment of malignancies, and diseases associated with neoangiogenesis (such as diabetic retinopathy and age-related macular degeneration).

The compositions in the invention may also be used in conjunction with and following processes or procedures involving maintaining blood vessel patency, including vascular surgery, vascular graft and stent patency, organ, tissue and cell implantation and transplantation.

The compositions in the invention may also be useful in the treatment of inflammatory diseases, septic shock and the vascular damage associated with infections.

The compositions of the invention are useful for the treatment of blood and blood products used in dialysis, blood storage in the fluid phase, especially ex vivo platelet aggregation. The present compositions may also be added to human plasma during the analysis of blood chemistry in hospital settings to determine the fibrinolytic capacity thereof.

The compositions in the present invention may also be used in combination with prothrombolytic, fibrinolytic and anticoagulant agents.

The compositions of the present invention may also be used to treat cancer including, but not limited to, breast and ovarian cancer, and as imaging agents for the identification of metastatic cancers.

The compositions of the invention may also be used in the treatment of Alzheimer's disease. This method may also be characterized as the inhibition of plasminogen activator by PAI-1 in a mammal, particularly a human, experiencing or subject to Alzheimer's disease. This method may also be characterized as a method of increasing or normalizing levels of plasmin concentration in a mammal, particularly those experiencing or subject to Alzheimer's disease.

The compositions of the invention maybe used for reducing amyloid beta levels in a mammal, preferably a human, suffering from Alzheimer's disease, comprising the administration of a therapeutically effective amount of the composition. In some embodiments, the methods of this invention reduce amyloid beta levels in the brain.

The compositions of the invention may be used for improving cognition in a mammal, preferably a human, comprising the administration of a therapeutically effective amount of the composition.

The compositions of the invention maybe used for treating pre-senile or senile dementia in a mammal, preferably a human.

The compositions of the invention are useful in the manufacture of a medicament useful for the treatment of Alzheimer's disease in a mammal, preferably a human.

The compositions of the invention may be used for the treatment of myelofibrosis with myeloid metaplasia by regulating stromal cell hyperplasia and increases in extracellular matrix proteins.

The compositions of the invention may also be used in conjunction with protease inhibitor-containing highly active antiretroviral therapy (HAART) for the treatment of diseases which originate from fibrinolytic impairment and hyper-coagulability of HIV-1 infected patients receiving such therapy.

The compositions of the invention may be used for the treatment of diabetic nephropathy and renal dialysis associated with nephropathy.

The compositions of the invention may be used to treat cancer, septicemia, obesity, insulin resistance, proliferative diseases such as psoriasis, improve coagulation homeostasis, treat cerebrovascular diseases, microvascular disease, hypertension, dementia, osteoporosis, arthritis, asthma, heart failure, arrhythmia, angina, as a hormone replacement agent, and for treating, preventing or reversing progression of atherosclerosis, Alzheimer's disease, osteoporosis, and osteopenia; reduce inflammatory markers, reducing C-reactive protein, or for preventing or treating low grade vascular inflammation, stroke, dementia, coronary heart disease, for primary and secondary prevention of myocardial infarction, stable and unstable angina, primary prevention of coronary events, secondary prevention of cardiovascular events, peripheral vascular disease, peripheral arterial disease, acute vascular syndromes, reduce the risk of undergoing a myocardial revascularization procedure, treat microvascular diseases such as nephropathy, neuropathy, retinopathy and nephrotic syndrome, hypertension, Type I and 2 diabetes and related diseases, hyperglycemia, hyperinsulinemia, malignant lesions, premalignant lesions, gastrointestinal malignancies, liposarcomas and epithelial tumors, proliferative diseases such as psoriasis, improve coagulation homeostasis, and/or endothelial function, and treat all forms of cerebrovascular diseases.

Methods for the treatment, inhibition, prevention or prophylaxis in a mammal of each of the conditions or maladies listed herein are part of the present invention. Each method comprises administering to a mammal in need thereof a pharmaceutically or therapeutically effective amount of a compound of this invention, or a pharmaceutically acceptable salt or ester form thereof. Where a method of treatment is referred to herein, that method will also cover the prevention or prophylaxis of the same disorder, disease or condition being treated.

Each of the methods described herein comprise administering to a mammal in need of such treatment a pharmaceutically effective amount of a compound of this invention, or a pharmaceutically acceptable salt or ester form thereof. It will be understood that a pharmaceutically effective amount of the compound will be at least the minimum amount necessary to provide an improvement in or prevent progression of the symptoms or underlying causation of the malady in question or to arrest, inhibit or lessen the onset of symptoms of the malady.

The compositions of this invention are suitable for various oral dosage delivery forms including tablets, capsules, lozenges and the like. In some embodiments, the compositions comprise a capsule. Dosage amounts vary in accord to the compound used, the age of the patient, the type of illness being treated, the age and condition of the patient and so forth. As a general matter, dose ranges of about 1.0 mg to 500 mg may be contemplated. In some embodiments, the dose ranges contemplated may be between about 2.5 mg and 200 mg.

### EXAMPLES

The following examples are to be considered non-limiting. For purposes of this invention, embodiments maybe combined to achieve additional embodiments. The compositions of this invention, at a minimum, comprise a compound of the invention and one or more surfactants. Representative formulations of the invention are listed below.

### Example 1

| Composition Ingredient | Amount |
|---|---|
| [1-(4-tert-Butylbenzyl)-5-(3-methylphenyl)-1H- | 3.33% to 33.33% |
| indol-3-yl](oxo)acetic acid | |
| Surfactant(s) | 1% to 25% |
| Additional Excipients | Remainder |

### Example 2

| Composition Ingredient | Amount |
|---|---|
| [1-(4-tert-Butylbenzyl)-5-(3-methylphenyl)-1H- | 3.33% to 33.33% |
| indol-3-yl](oxo)acetic acid | |
| Surfactant(s) | 5% to 20% |
| Additional Excipients | Remainder |

### Example 3

| Composition Ingredient | Amount |
|---|---|
| [1-(4-tert-Butylbenzyl)-5-(3-methylphenyl)-1H- | 3.33% to 33.33% |
| indol-3-yl](oxo)acetic acid | |
| Surfactant(s) | 10% |
| Additional Excipients | Remainder |

### Example 4

| Composition Ingredient | Amount |
|---|---|
| [1-(4-tert-Butylbenzyl)-5-(3-methylphenyl)-1H- | 3.33% to 33.33% |
| indol-3-yl](oxo)acetic acid | |
| TPGS and/or sodium lauryl sulfate | 10% |
| Additional Excipients | Remainder |

### Example 5

| Composition Ingredient | Amount |
|---|---|
| [1-(4-tert-Butylbenzyl)-5-(3-methylphenyl)-1H- | 3.33% to 33.33% |
| indol-3-yl](oxo)acetic acid | |
| TPGS | 5% |
| Sodium lauryl sulfate | 5% |
| Additional Excipients | Remainder |

### Example 6

| Composition Ingredient | Amount |
|---|---|
| [1-(4-tert-Butylbenzyl)-5-(3-methylphenyl)-1H- | 3.33% to 33.33% |
| indol-3-yl](oxo)acetic acid | |
| Surfactant(s) | 1% to 25% |
| Binder(s) | 1% to 35% |
| Additional Excipients | Remainder |

### Example 7

| Composition Ingredient | Amount |
|---|---|
| [1-(4-tert-Butylbenzyl)-5-(3-methylphenyl)-1H- | 3.33% to 33.33% |
| indol-3-yl](oxo)acetic acid | |
| Surfactant(s) | 1% to 25% |
| Binder(s) | 5% to 25% |
| Additional Excipients | Remainder |

### Example 8

| Composition Ingredient | Amount |
|---|---|
| [1-(4-tert-Butylbenzyl)-5-(3-methylphenyl)-1H- | 3.33% to 33.33% |
| indol-3-yl](oxo)acetic acid | |
| Surfactant(s) | 1% to 25% |
| Binder(s) | 10% to 20% |
| Additional Excipients | Remainder |

### Example 9

| Composition Ingredient | Amount |
|---|---|
| [1-(4-tert-Butylbenzyl)-5-(3-methylphenyl)-1H- | 3.33% to 33.33% |
| indol-3-yl](oxo)acetic acid | |
| Surfactant(s) | 10% |
| Binder(s) | 10% to 20% |
| Additional Excipients | Remainder |

### Example 10

| Composition Ingredient | Amount |
|---|---|
| [1-(4-tert-Butylbenzyl)-5-(3-methylphenyl)-1H- | 3.33% to 33.33% |
| indol-3-yl](oxo)acetic acid | |
| TPGS and/or sodium lauryl sulfate | 10% |
| Binder(s) | 10% to 20% |
| Additional Excipients | Remainder |

### Example 11

| Composition Ingredient | Amount |
|---|---|
| [1-(4-tert-Butylbenzyl)-5-(3-methylphenyl)-1H- | 3.33% to 33.33% |
| indol-3-yl](oxo)acetic acid | |
| TPGS and/or sodium lauryl sulfate | 10% |
| Microcrystalline cellulose | 10% to 20% |
| Additional Excipients | Remainder |

### Example 12

| Composition Ingredient | Amount |
|---|---|
| [1-(4-tert-Butylbenzyl)-5-(3-methylphenyl)-1H- | 3.33% to 33.33% |
| indol-3-yl](oxo)acetic acid | |
| TPGS and/or sodium lauryl sulfate | 10% |
| Microcrystalline cellulose | 15% |
| Additional Excipients | Remainder |

### Example 13

| Composition Ingredient | Amount |
|---|---|
| [1-(4-tert-Butylbenzyl)-5-(3-methylphenyl)-1H- | 3.33% to 33.33% |
| indol-3-yl](oxo)acetic acid | |
| TPGS | 5% |
| Sodium lauryl sulfate | 5% |
| Microcrystalline cellulose | 15% |
| Additional Excipients | Remainder |

### Example 14

| Composition Ingredient | Amount |
|---|---|
| [1-(4-tert-Butylbenzyl)-5-(3-methylphenyl)-1H- | 3.33% to 33.33% |
| indol-3-yl](oxo)acetic acid | |
| Surfactant(s) | 1% to 25% |
| Binder(s) | 1 to 35% |
| Disintegrant(s) | 1 to 15% |
| Additional Excipients | Remainder |

### Example 15

| Composition Ingredient | Amount |
|---|---|
| [1-(4-tert-Butylbenzyl)-5-(3-methylphenyl)-1H- | 3.33% to 33.33% |
| indol-3-yl](oxo)acetic acid | |
| Surfactant(s) | 1% to 25% |
| Binder(s) | 1 to 35% |
| Disintegrant(s) | 2 to 8% |
| Additional Excipients | Remainder |

### Example 16

| Composition Ingredient | Amount |
|---|---|
| [1-(4-tert-Butylbenzyl)-5-(3-methylphenyl)-1H- | 3.33% to 33.33% |
| indol-3-yl](oxo)acetic acid | |
| Surfactant(s) | 1% to 25% |
| Binder(s) | 1 to 35% |
| Croscarmellose Sodium | 1 to 15% |
| Additional Excipients | Remainder |

### Example 17

| Composition Ingredient | Amount |
|---|---|
| [1-(4-tert-Butylbenzyl)-5-(3-methylphenyl)-1H- | 3.33% to 33.33% |
| indol-3-yl](oxo)acetic acid | |
| Surfactant(s) | 1% to 25% |
| Binder(s) | 1 to 35% |
| Croscarmellose Sodium | 2 to 8% |
| Additional Excipients | Remainder |

### Example 18

| Composition Ingredient | Amount |
|---|---|
| [1-(4-tert-Butylbenzyl)-5-(3-methylphenyl)-1H- | 3.33% to 33.33% |
| indol-3-yl](oxo)acetic acid | |
| Surfactant(s) | 1% to 25% |
| Binder(s) | 1 to 35% |
| Croscarmellose Sodium | 5% |
| Additional Excipients | Remainder |

### Example 19

| Composition Ingredient | Amount |
|---|---|
| [1-(4-tert-Butylbenzyl)-5-(3-methylphenyl)-1H- | 3.33% to 33.33% |
| indol-3-yl](oxo)acetic acid | |
| TPGS | 5% |
| Sodium lauryl sulfate | 5% |
| Microcrystalline Cellulose | 15% |
| Croscarmellose Sodium | 5% |
| Additional Excipients | Remainder |

### Example 20

| Composition Ingredient | Amount |
|---|---|
| [1-(4-tert-Butylbenzyl)-5-(3-methylphenyl)-1H- | 3.33% to 33.33% |
| indol-3-yl](oxo)acetic acid | |
| Surfactant(s) | 1% to 25% |
| Binder(s) | 1 to 35% |
| Disintegrant(s) | 2 to 8% |
| Glidant | 0.01% to 5% |
| Additional Excipients | Remainder |

### Example 21

| Composition Ingredient | Amount |
|---|---|
| [1-(4-tert-Butylbenzyl)-5-(3-methylphenyl)-1H- | 3.33% to 33.33% |
| indol-3-yl](oxo)acetic acid | |
| Surfactant(s) | 1% to 25% |
| Binder(s) | 1 to 35% |
| Disintegrant(s) | 2 to 8% |
| Glidant | 0.1% to 3% |
| Additional Excipients | Remainder |

### Example 22

| Composition Ingredient | Amount |
|---|---|
| [1-(4-tert-Butylbenzyl)-5-(3-methylphenyl)-1H- | 3.33% to 33.33% |
| indol-3-yl](oxo)acetic acid | |
| Surfactant(s) | 1% to 25% |
| Binder(s) | 1 to 35% |
| Disintegrant(s) | 2 to 8% |
| Glidant | 0.01% to 1% |
| Additional Excipients | Remainder |

### Example 23

| Composition Ingredient | Amount |
|---|---|
| [1-(4-tert-Butylbenzyl)-5-(3-methylphenyl)-1H- | 3.33% to 33.33% |
| indol-3-yl](oxo)acetic acid | |
| Surfactant(s) | 1% to 25% |
| Binder(s) | 1 to 35% |
| Disintegrant(s) | 2 to 8% |
| Glidant | 0.1% to 1% |
| Additional Excipients | Remainder |

### Example 24

| Composition Ingredient | Amount |
|---|---|
| [1-(4-tert-Butylbenzyl)-5-(3-methylphenyl)-1H- | 3.33% to 33.33% |
| indol-3-yl](oxo)acetic acid | |
| Surfactant(s) | 1% to 25% |
| Binder(s) | 1 to 35% |
| Disintegrant(s) | 2 to 8% |
| Glidant | 0.5% |
| Additional Excipients | Remainder |

### Example 25

| Composition Ingredient | Amount |
|---|---|
| [1-(4-tert-Butylbenzyl)-5-(3-methylphenyl)-1H- | 3.33% to 33.33% |
| indol-3-yl](oxo)acetic acid | |
| Surfactant(s) | 1% to 25% |
| Binder(s) | 1 to 35% |
| Disintegrant(s) | 2 to 8% |
| Colloidal Silicon Dioxide | 0.5% |
| Additional Excipients | Remainder |

### Example 26

| Composition Ingredient | Amount |
|---|---|
| [1-(4-tert-Butylbenzyl)-5-(3-methylphenyl)-1H- | 3.33% |
| indol-3-yl](oxo)acetic acid | |
| TPGS | 5% |
| Sodium Lauryl Sulfate | 5% |
| Microcrystalline Cellulose | 15% |
| Croscarmellose Sodium | 2% to 8% |
| Colloidal Silicon Dioxide | 0.5% |
| Additional Excipients | Remainder |

### Example 27

| Composition Ingredient | Amount |
|---|---|
| [1-(4-tert-Butylbenzyl)-5-(3-methylphenyl)-1H- | 3.33% to 33.33% |
| indol-3-yl](oxo)acetic acid | |
| TPGS | 5% |
| Sodium Lauryl Sulfate | 5% |
| Microcrystalline Cellulose | 15% |
| Croscarmellose Sodium | 2% to 8% |
| Colloidal Silicon Dioxide | 0.5% |
| Additional Excipients | Remainder |

### Example 28

| Composition Ingredient | Amount |
|---|---|
| [1-(4-tert-Butylbenzyl)-5-(3-methylphenyl)-1H- | 3.33% to 33.33% |
| indol-3-yl](oxo)acetic acid | |
| Surfactant(s) | 1% to 25% |
| Binder(s) | 1 to 35% |
| Disintegrant(s) | 2 to 8% |
| Glidant | 0.01% to 5% |
| Lubricant | 0.01% to 5% |
| Additional Excipients | Remainder |

### Example 29

| Composition Ingredient | Amount |
|---|---|
| [1-(4-tert-Butylbenzyl)-5-(3-methylphenyl)-1H- | 3.33% to 33.33% |
| indol-3-yl](oxo)acetic acid | |
| Surfactant(s) | 1% to 25% |
| Binder(s) | 1 to 35% |
| Disintegrant(s) | 2 to 8% |
| Glidant | 0.01% to 5% |
| Lubricant | 0.1% to 5% |
| Additional Excipients | Remainder |

### Example 30

| Composition Ingredient | Amount |
|---|---|
| [1-(4-tert-Butylbenzyl)-5-(3-methylphenyl)-1H- | 3.33% to 33.33% |
| indol-3-yl](oxo)acetic acid | |
| Surfactant(s) | 1% to 25% |
| Binder(s) | 1 to 35% |
| Disintegrant(s) | 2 to 8% |
| Glidant | 0.01% to 5% |
| Lubricant | 0.1% to 1% |
| Additional Excipients | Remainder |

### Example 31

| Composition Ingredient | Amount |
|---|---|
| [1-(4-tert-Butylbenzyl)-5-(3-methylphenyl)-1H- | 3.33% to 33.33% |
| indol-3-yl](oxo)acetic acid | |
| Surfactant(s) | 1% to 25% |
| Binder(s) | 1 to 35% |
| Disintegrant(s) | 2 to 8% |
| Glidant | 0.01% to 5% |
| Lubricant | 0.5% |
| Additional Excipients | Remainder |

### Example 32

| Composition Ingredient | Amount |
|---|---|
| [1-(4-tert-Butylbenzyl)-5-(3-methylphenyl)-1H- | 3.33% to 33.33% |
| indol-3-yl](oxo)acetic acid | |
| Surfactant(s) | 1% to 25% |
| Binder(s) | 1 to 35% |
| Disintegrant(s) | 2 to 8% |
| Glidant | 0.01% to 5% |
| Magnesium Stearate | 0.5% |
| Additional Excipients | Remainder |

### Example 33

| Composition Ingredient | Amount |
|---|---|
| [1-(4-tert-Butylbenzyl)-5-(3-methylphenyl)-1H- | 3.33% to 33.33% |
| indol-3-yl](oxo)acetic acid | |
| TPGS | 5% |
| Sodium Lauryl Sulfate | 5% |
| Microcrystalline Cellulose | 15% |
| Croscarmellose Sodium | 5% |
| Colloidal Silicon Dioxide | 0.5% |
| Magnesium Stearate | 0.5% |
| Additional Excipients | Remainder |

### Example 34

| Composition Ingredient | Amount |
|---|---|
| [1-(4-tert-Butylbenzyl)-5-(3-methylphenyl)-1H- | 3.33% to 33.33% |
| indol-3-yl](oxo)acetic acid | |
| TPGS | 5% |
| Sodium Lauryl Sulfate | 5% |
| Microcrystalline Cellulose | 15% |
| Croscarmellose Sodium | 5% |
| Colloidal Silicon Dioxide | 0.5% |
| Magnesium Stearate | 0.5% |
| Filler | Remainder |

### Example 35

| Composition Ingredient | Amount |
|---|---|
| [1-(4-tert-Butylbenzyl)-5-(3-methylphenyl)-1H- | 3.33% |
| indol-3-yl](oxo)acetic acid | |
| TPGS | 5% |
| Sodium Lauryl Sulfate | 5% |
| Microcrystalline Cellulose | 15% |
| Croscarmellose Sodium | 5% |
| Colloidal Silicon Dioxide | 0.5% |
| Magnesium Stearate | 0.5% |
| Mannitol | 65.67% |

Fill Weight of 300 mg for 10 mg Active Ingredient Capsule
Fill Weight of 75 mg for 2.5 mg Active Ingredient Capsule

### Example 36

10 mg or 2.5 mg Active Ingredient Capsules

| Composition Ingredient | Amount |
|---|---|
| [1-(4-tert-Butylbenzyl)-5-(3-methylphenyl)-1H- | 3.33% |
| indol-3-yl](oxo)acetic acid | |
| Sodium Lauryl Sulfate | 5% |
| Microcrystalline Cellulose | 15% |
| Croscarmellose Sodium | 5% |
| Colloidal Silicon Dioxide | 0.5% |
| Magnesium Stearate | 0.5% |
| Mannitol | 70.67% |

Fill Weight of 300 mg for 10 mg Active Ingredient Capsule
Fill Weight of 75 mg for 2.5 mg Active Ingredient Capsule

### Example 37

| Composition Ingredient | Amount |
|---|---|
| [1-(4-tert-Butylbenzyl)-5-(3-methylphenyl)-1H- | 33.33% |
| indol-3-yl](oxo)acetic acid | |
| TPGS | 5% |
| Sodium Lauryl Sulfate | 5% |
| Microcrystalline Cellulose | 15% |
| Croscarmellose Sodium | 5% |
| Colloidal Silicon Dioxide | 0.5% |
| Magnesium Stearate | 0.5% |
| Mannitol | 35.67% |

Fill Weight of 300 mg for 100 mg Active Ingredient Capsule
Fill Weight of 75 mg for 25 mg Active Ingredient Capsule

### Example 38

| Composition Ingredient | Amount |
|---|---|
| [1-(4-tert-Butylbenzyl)-5-(3-methylphenyl)-1H- | 33.33% |
| indol-3-yl](oxo)acetic acid | |
| Sodium Lauryl Sulfate | 5% |
| Microcrystalline Cellulose | 15% |
| Croscarmellose Sodium | 5% |
| Colloidal Silicon Dioxide | 0.5% |
| Magnesium Stearate | 0.5% |
| Mannitol | 40.67% |

Fill Weight of 300 mg for 100 mg Active Ingredient Capsule
Fill Weight of 75 mg for 25 mg Active Ingredient Capsule

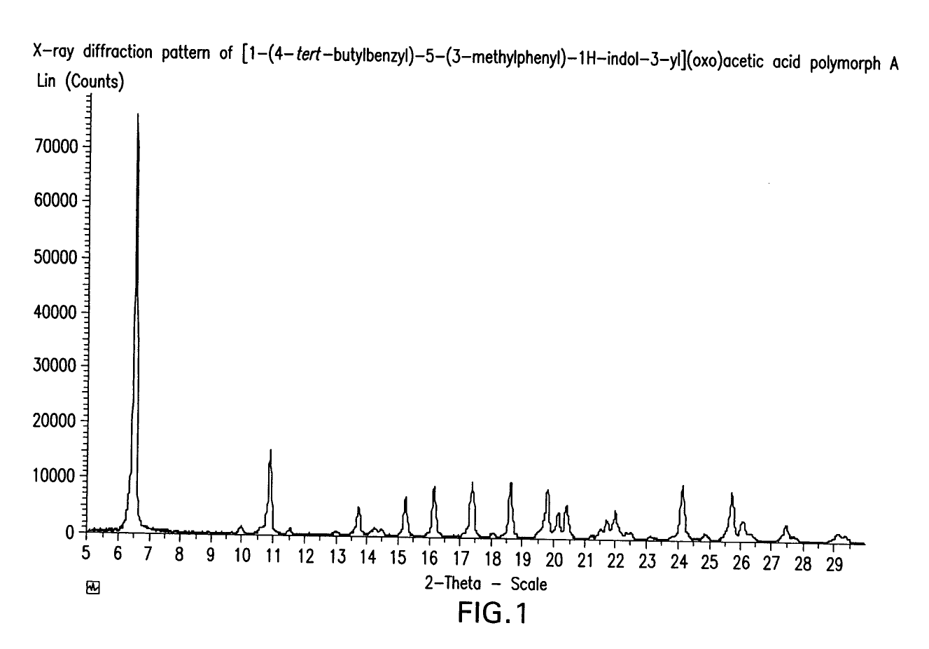

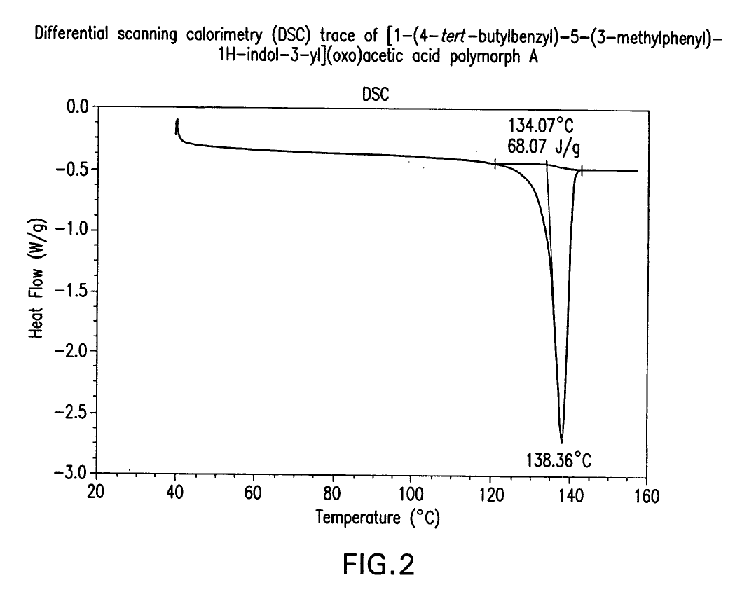

In some embodiments, the compositions of the invention comprise the compound, [1-(4-tert-butylbenzyl)-5-(3-methylphenyl)-1H-indol-3-yl](oxo)acetic acid, which is polymorphic as described U.S. Provisional Patent Application No. 60/899,473, filed Feb. 5, 2007, titled “Novel Indole Polymorphs,” the entire disclosure of which is incorporated by reference herein. Preferably, the [1-(4-tert-butylbenzyl)-5-(3-methylphenyl)-1H-indol-3-yl](oxo)acetic acid is micronized prior to use in preparing the formulations. In some embodiments, the compounds of the invention are preferably prepared using the polymorph of [1-(4-tert-butylbenzyl)-5-(3-methylphenyl)-1H-indol-3-yl](oxo)acetic acid desiganted as “Form A”, which has the X-ray diffraction spectrum shown in FIG. 1, and summarized in the Table 1, below, and the differential scanning calorimetry (DSC) trace shown in FIG. 2.

TABLE 1
Powder diffraction data for [1-(4-tert-butylbenzyl)-5-
(3-methylphenyl)-1H-indol-3-yl](oxo)acetic acid Polymorph A

| | Intensity, |
|---|---|
| Degree (2θ) | (% of the largest peak size) |
| 6.5 | 100.0 |
| 10.9 | 19.9 |
| 18.6 | 13.6 |
| 24.2 | 13.6 |
| 17.4 | 13.0 |
| 16.2 | 12.2 |
| 25.8 | 10.8 |
| 15.2 | 9.5 |
| 19.8 | 9.4 |
| 20.4 | 8.9 |
| 22.0 | 7.3 |
| 20.1 | 6.6 |
| 13.7 | 6.4 |
| 21.7 | 4.7 |
| 26.1 | 4.2 |
| 27.5 | 3.2 |
| 14.5 | 2.1 |
| 9.9 | 1.7 |
| 11.5 | 1.5 |
| 24.9 | 1.2 |
| 14.2 | 1.2 |

### Preparation of Formulations

The compounds useful in the compositions of this invention can be prepared according to the procedures described in U.S. application No. 2003/0125371, now U.S. Pat. No. 7,074,817, each of which is herein incorporated by reference in its entirety.

The compositions of this invention may be prepared according to various methods known to those of skill in the art. The following examples are not to be construed as limiting the invention to any particular process of preparation nor any particular oral dosage form.

According to the methods of this invention, the formulations of this invention maybe prepared by any method known to those of ordinary skill in the art. Ingredients for use in the formulations of this invention may be dry blended or wet blended. Individual or groups of components may be first dry blended and then wet blended together, thus the processes for the preparation of the formulations of this invention are contemplated to include mixed blending regimens. The formulations of this invention may also be prepared by, for example, a melt granulation where two or more ingredients are combined and then melted together and then further processed.

The preparation of two representative formulations of the invention are shown below. However, the formulations of this invention are not to be construed or limited by the processes specifically delineated herein but rather include any and all processes ascertainable by one of ordinary skill in the art.

### Preparation of Formulation of Example 37

1. Prepared a 15% w/w aqueous solution of TPGS suitable for wet granulation by adding the appropriate weight of TPGS to purified water and heating to approximately 50° C. with stirring.
2. Blended micronized [1-(4-tert-Butylbenzyl)-5-(3-methylphenyl)-1H-indol-3-yl](oxo)acetic acid, mannitol, microcrystalline cellulose, croscarmellose sodium and sodium lauryl sulfate.
3. Slowly added the TPGS (aq) solution to the powder blend with stirring.
4. Wet screened and dried the granule.
5. Screened the dried granule.
6. Added colloidal silicon dioxide to the dried granule and blend.
7. Lubricated the dried granule with 0.5% w/w magnesium stearate.
8. Encapsulated the granule to the required fill weight into suitability sized HPMC or gelatin capsules.
9. Alternatively, compress the granule to form a tablet.

### Preparation of Formulation of Example 38

1. Blended micronized [1-(4-tert-Butylbenzyl)-5-(3-methylphenyl)-1H-indol-3-yl](oxo)acetic acid, mannitol, microcrystalline cellulose, crocarmellose sodium, and sodium lauryl sulfate.
2. Slowly added purified water to the mix.
3. Screen the dried granule.
4. Wet screened and dried the granule.
5. Added colloidal silicon dioxide to the dried granule and blend.
6. Lubricated the dried granule with 0.5% (w/w) magnesium stearate.
7. Encapsulated the granule to the required fill weight into suitably sized HPMC or gelatin capsules.
8. Alternatively, compress the granules to form a tablet.

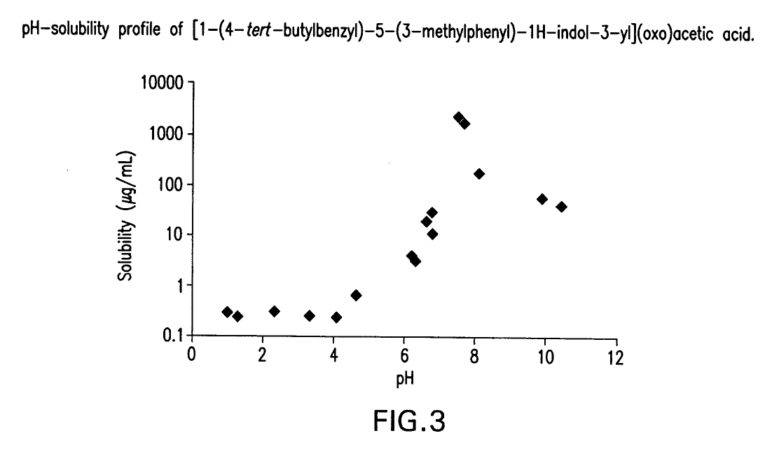

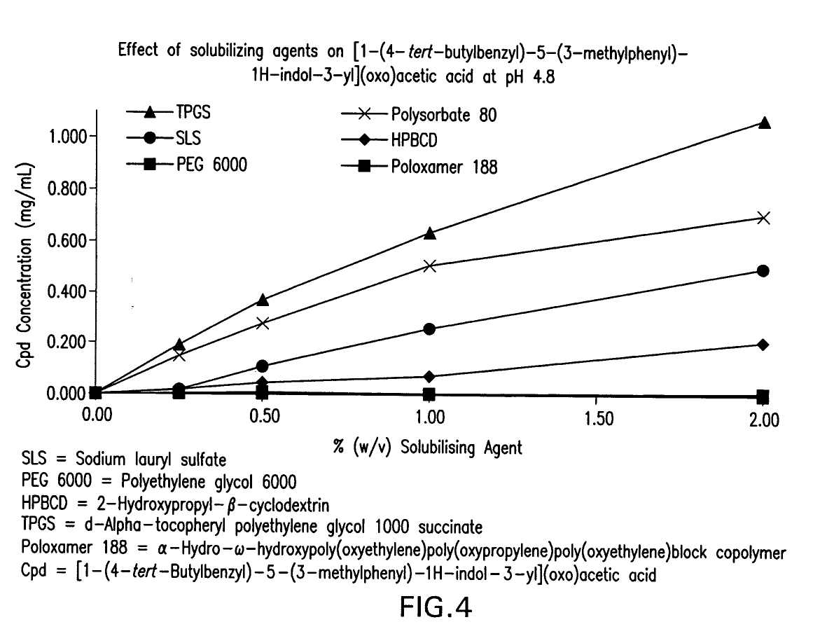

### Solubility Profile

[1-(4-tert-Butylbenzyl)-5-(3-methylphenyl)-1H-indol-3-yl](oxo)acetic acid is an α-oxo carboxylic acid with a calculated pKa of 3.53 for the indole oxoacetic acid, and an aqueous solubility of approximately 0.25 uG/mL in the ionized form which increases to approximately 24 uG/mL upon ionization in aqueous media. A solubility profile for [1-(4-tert-butylbenzyl)-5-(3-methylphenyl)-1H-indol-3-yl](oxo)acetic acid was generated using the free acid in HCl/NaOH solution (See FIG. 3). Solutions were centrifuged after 24 hours equilibration (2 hours for pH<4 to avoid degradation) and the supernatants were assayed by HPLC. Above pH 4, solubility increases with increasing pH up to pH-8. Above pH 8, solubility decreases due to precipitation of the sodium salt, which is of very small particle size. Throughout the pH range, samples remained “cloudy” after filtration through a 0.2 μfilter, hence centrifugation for 2 hours was used as a means of phase separation. The low solubility at acidic pH's indicates that dissolution of [1-(4-tert-Butylbenzyl)-5-(3-methylphenyl)-1H-indol-3-yl](oxo)acetic acid will not occur appreciably in the stomach, but rather in the small intestine as the pH reaches near-neutral values. Because of the poor solubility at pH<4, the stomach condition, a number of surfactant excipients were examined for their ability to increase the solubility of the compounds of the compositions of the invention, as shown in FIG. 4.

### Dissolution Studies

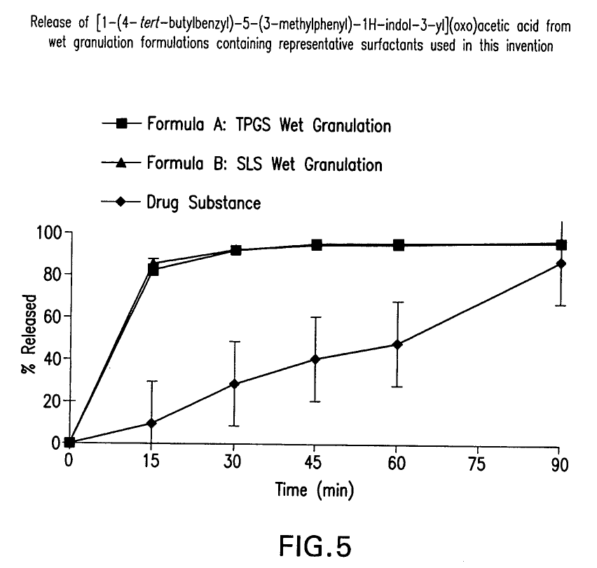

Dissolution studies were performed. FIG. 5 demonstrates a much rapid release of [1-(4-tert-butylbenzyl)-5-(3-methylphenyl)-1H-indol-3-yl](oxo)acetic acid with the surfactant TPGS or SLS presenting in the formulations. Table 2 shows the compositions of the two [1-(4-tert-butylbenzyl)-5-(3-methylphenyl)-1H-indol-3-yl](oxo)acetic acid wet granulation formulations containing TPGS or SLS. Dissolution medium was sodium phosphate buffer (pH 6.8) with 0.01% (w/v) Tween 80. Paddles were set at 50 RPM, and increased to 250 RPM after 60 minutes.

TABLE 2
Wet granulation formulations containing one
or two of the representative surfactants

| | Formula A | Formula B |
|---|---|---|
| Component | % (w/w) | % (w/w) |
| [1-(4-tert-Butylbenzyl)-5-(3-methylphenyl)- | 33.3 | 33.3 |
| 1H-indol-3-yl](oxo)acetic acid | | |
| Mannitol 100 SD | 41.2 | 40.7 |
| Microcrystalline Cellulose | 15.0 | 15.0 |
| Croscarmellose Sodium | 5.0 | 5.0 |
| TPGS | 5.0 | 0 |
| SLS | 0 | 5.0 |
| Colloidal Silicon Dioxide | 0 | 0.5 |
| Magnesium Stearate | 0.5 | 0.5 |
| Total | 100 | 100 |

### Bioavailability

The formulations of Example 37 and Example 38 were evaluated in Beagle dogs. To each group of 4 dogs, a single oral dose was administered after an overnight fast and 30 minutes following the administration of food (standard chow). Blood samples were drawn at 0 (predose), 0.5, 1, 2, 3, 4, 6, 8, 12 and 24 hours after dosing, plasma was separated and assayed for [1-(4-tert-butylbenzyl)-5-(3-methylphenyl)-1H-indol-3-yl](oxo)acetic acid content.

For PK assessment, individual dog plasma [1-(4-tert-Butylbenzyl)-5-(3-methylphenyl)-1H-indol-3-yl](oxo)acetic acid concentration-time profiles were subjected to noncompartmental pharmacokinetic analyses (WinNonlin, Model 200). The pharmacokinetic parameters, AUC, Cₘₐₓ, tₘₐₓ and t_{1/2}, were determined for each dog, and descriptive statistics were calculated for comparison among formulations. The formulation of Example 37 containing TPGS dramatically increased the AUC of the fasted dogs and reduced food effect as compared to the formulation of Example 38. Results are tabulated in Table 3. It was noted that the formulation containing both TPGS and SLS surfactants resulted in a much greater fasted AUC and lower food effect compared to that containing only SLS.

TABLE 3
Pharmacokinetic Parameters in Male Dogs Following Single Oral Dose
of 100 mg [1-(4-tert-Butylbenzyl)-5-(3-methylphenyl)-1H-indol-3-
yl](oxo)acetic acid Capsules

| | Formula (Example 37) | | | Formula (Example 38) | | |
|---|---|---|---|---|---|---|
| | Fed | Fasted | Fed/Fasted | Fed | Fasted | Fed/Fasted |
| Dose | 10.6 | 10.6 | | 9.26 | 9.26 | |
| (mg/kg) | 8.77 | 8.93 | | 10.0 | 10.0 | |
| | 8.93 | 8.77 | | 11.4 | 10.6 | |
| | 8.47 | 8.62 | | 10.6 | 10.2 | |
| Mean | 9.20 | 9.24 | | 10.3 | 10.0 | |
| SD | 0.97 | 0.94 | | 0.90 | 0.58 | |
| AUC_{0-∞} | 44237 | 36613 | 1.21 | 50855 | 15886 | 3.20 |
| (ng · hr/mL) | 38626 | 54559 | 0.71 | 59050 | 26512 | 2.23 |
| | 131609 | 30431 | 4.32 | 33246 | 14661 | 2.27 |
| | 97436 | 33676 | 2.89 | 161901 | 35628 | 4.54 |
| Mean | 77977 | 38820 | 2.28 | 76263 | 23172 | 3.06 |
| SD | 44504 | 10792 | 1.65 | 58098 | 9863 | 1.09 |
| AUC/Dose | 4158 | 3442 | 1.21 | 5492 | 1716 | 3.20 |
| | 4403 | 6111 | 0.72 | 5905 | 2651 | 2.23 |
| | 14740 | 3469 | 4.25 | 2926 | 1378 | 2.12 |
| | 11497 | 3906 | 2.94 | 15219 | 3492 | 4.36 |
| Mean | 8700 | 4232 | 2.28 | 7385 | 2309 | 2.98 |
| SD | 5273 | 1270 | 1.62 | 5386 | 955 | 1.04 |
| Cₘₐₓ | 4963 | 4205 | 1.18 | 9494 | 3447 | 2.75 |
| (ng/mL) | 5353 | 8132 | 0.66 | 10440 | 4226 | 2.47 |
| | 18103 | 4786 | 3.78 | 6965 | 3193 | 2.18 |
| | 13405 | 4326 | 3.10 | 9932 | 3156 | 3.15 |
| Mean | 10456 | 5363 | 2.18 | 9208 | 3506 | 2.64 |
| SD | 6413 | 1863 | 1.50 | 1544 | 497 | 0.41 |
| Cₘₐₓ/Dose | 467 | 395 | 1.18 | 1025 | 372 | 2.75 |
| | 610 | 911 | 0.67 | 1044 | 423 | 2.47 |
| | 2028 | 546 | 3.72 | 613 | 300 | 2.04 |
| | 1582 | 502 | 3.15 | 934 | 309 | 3.02 |
| Mean | 1172 | 588 | 2.18 | 904 | 351 | 2.57 |
| SD | 756 | 224 | 1.48 | 200 | 57.4 | 0.42 |
| tₘₐₓ | 2.00 | 2.00 | | 2.00 | 1.00 | |
| (hr) | 3.00 | 2.00 | | 1.00 | 2.00 | |
| | 3.00 | 2.00 | | 1.00 | 1.00 | |
| | 2.00 | 2.00 | | 3.00 | 4.00 | |
| Mean | 2.50 | 2.00 | | 1.75 | 2.00 | |
| SD | 0.58 | 0.00 | | 0.96 | 1.41 | |
| t_{1/2} | 5.42 | 4.64 | | 3.93 | 3.48 | |
| (hr) | 4.61 | 3.81 | | 4.45 | 2.96 | |
| | 5.74 | 4.36 | | 3.29 | 3.02 | |
| | 5.92 | 4.15 | | 7.86 | 6.40 | |
| Mean | 5.42 | 4.24 | | 4.88 | 3.96 | |
| SD | 0.58 | 0.35 | | 2.04 | 1.64 | |

This application claims the benefit of U.S. Provisional Patent Application No. 60/899,575, filed Feb. 5, 2007, the entire disclosure of which is incorporated by reference herein.

It should be appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

Those skilled in the art will recognize that various changes and/or modifications may be made to aspects or embodiments of this invention and that such changes and/or modifications may be made without departing from the spirit of this invention. Therefore, it is intended that the appended claims cover all such equivalent variations as will fall within the spirit and scope of this invention. It is intended that each of the patents, applications, and printed publications, including books, mentioned in this patent document be hereby incorporated by reference in their entirety.

What is claimed is:

1. A composition comprising a compound of formula (I), or a pharmaceutically acceptable salt, solvate or ester thereof: 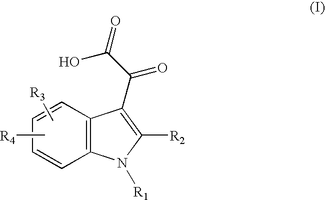
wherein:
R₁ is selected from C₁-C₈ alkyl, (—CH₂)ₙ—C₃-C₆ cycloalkyl, wherein n is an integer of from 0 to 3, pyridinyl, —CH₂-pyridinyl, phenyl or benzyl, the rings of the cycloalkyl, pyridinyl, phenyl and benzyl groups being optionally substituted by, from 1 to 3 groups independently selected from halogen, C₁-C₄ alkyl, C₁-C₃ perfluoroalkyl, —O—C₁-C₃ perfluoroalkyl, C₁-C₃ alkoxy, —OH, —NH₂, and —NO₂;
R₂ is selected from H, C₁-C₆ alkyl, C₃-C₆ cycloalkyl, —CH₂—C₃-C₆ cycloalkyl, or C₁-C₃ perfluoroalkyl, —CH₂OH or CH₂OAc;
R₃ is selected from H, halogen, C₁-C₆ alkyl, C₁-C₃ perfluoroalkyl, C₁-C₆ alkoxy, C₃-C₆ cycloalkyl, —CH₂—C₃-C₆ cycloalkyl, C₃-C₆ cycloalkenyl, —CH₂—C₃-C₆ cycloalkenyl, —NH₂, or —NO₂;
R₄ is phenyl substituted by from 1 to 3 groups independently selected from halogen, C₁-C₄ alkyl, C₁-C₃ alkoxy, C₁-C₃ perfluoroalkyl, and —O—C₁-C₃ perfluoroalkyl; and
from 1% to 25% w/w of one or more surfactants.

2. The composition of claim 1, wherein the compound of formula (I) is a compound of formula (II) or (III), or a pharmaceutically acceptable salt, solvate or ester thereof:

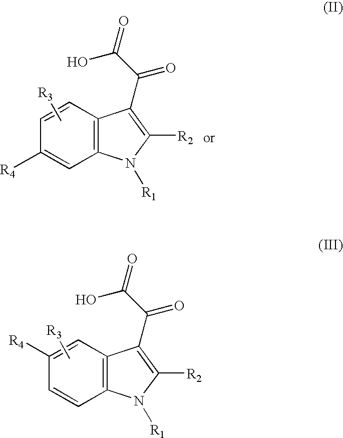

3. The composition of claim 2, wherein the compound of formula (I) is a compound of formula (IV) or formula (V), or a pharmaceutically acceptable salt, solvate or ester thereof: 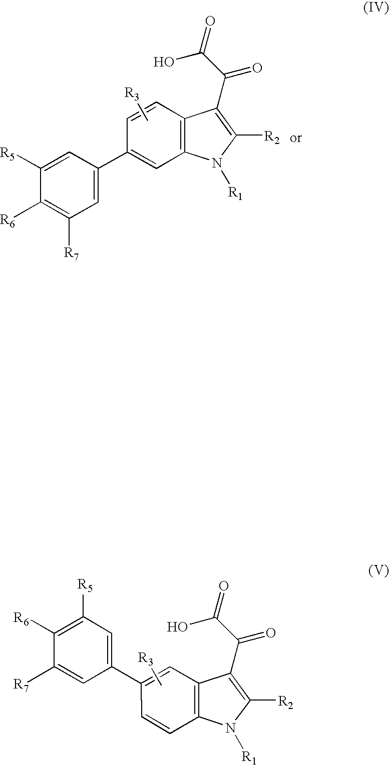
wherein:
R₁ is selected from C₁-C₈ alkyl, C₃-C₆ cycloalkyl, —CH₂—C₃-C₆ cycloalkyl, or benzyl, the rings of the cycloalkyl and benzyl groups being optionally substituted by from 1 to 3 groups selected from halogen, C₁-C₄ alkyl, C₁-C₃ perfluoroalkyl, —O—C₁-C₃ perfluoroalkyl, C₁-C₃ alkoxy, —OH, —NH₂, or —NO₂;
R₂ is selected from H, C₁-C₆ alkyl, C₃-C₆ cycloalkyl, —CH₂—C₃-C₆ cycloalkyl, or C₁-C₃ perfluoroalkyl;
R₃ is selected from H, halogen, C₁-C₆ alkyl, C₁-C₃ perfluoroalkyl, C₁-C₆ alkoxy, C₃-C₆ cycloalkyl, —CH₂—C₃-C₆ cycloalkyl, —NH₂, or —NO₂; and
R₅, R₆ and R₇ are independently selected from H, halogen, C₁-C₄ alkyl, C₁-C₃ perfluoroalkyl, —O—C₁-C₃ perfluoroalkyl, and C₁-C₃ alkoxy, provided that at least one of R₅, R₆ and R₇ is not H.

4. The composition of claim 3, wherein the compound of formula (I) is a compound of formula (VI), or a pharmaceutically acceptable salt, solvate or ester thereof: 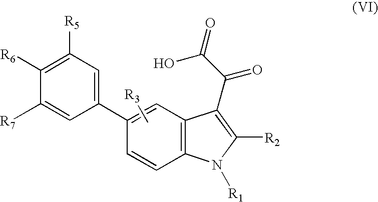
wherein:
R₁ is selected from benzyl, the benzyl group being optionally substituted by from 1 to 3 groups independently selected from halogen, C₁-C₄ alkyl, C₁-C₃ perfluoroalkyl, —O—C₁-C₃ perfluoroalkyl, and C₁-C₃ alkoxy;
R₂ is H;
R₃ is H; and
R₅, R₆ and R₇ are independently selected from H, halogen, C₁-C₄ alkyl, C₁-C₃ perfluoroalkyl, —O—C₁-C₃ perfluoroalkyl and C₁-C₃ alkoxy, provided that at least one of R₅, R₆ and R₇ is not H.

5. The composition of claim 1, wherein the compound of formula (I) is:
a) {1-Methyl-6-[4-(trifluoromethoxy)phenyl]-1H-indol-3-yl}(oxo)acetic acid;
b) {1-Methyl-6-[4-(trifluoromethyl)phenyl]-1H-indol-3-yl}(oxo)acetic acid;
c) {1-Ethyl-6-[4-(trifluoromethoxy)phenyl]-1H-indol-3-yl}(oxo)acetic acid;
d) {1-Ethyl-6-[4-(trifluoromethyl)phenyl]-1H-indol-3-yl}(oxo)acetic acid;
e) {1-Benzyl-6-[4-(trifluoromethoxy)phenyl]-1H-indol-3-yl}(oxo)acetic acid;
f) {1-Benzyl-6-[4-(trifluoromethyl)phenyl]-1H-indol-3-yl}(oxo)acetic acid;
g) {1-[4-(tert-Butyl)benzyl]-6-[4-(trifluoromethyl)phenyl]-1H-indol-3-yl}(oxo)acetic acid;
h) {1-[4-(tert-Butyl)benzyl]-6-[4-(trifluoromethoxy)phenyl]-1H-indol-3-yl}(oxo)acetic acid;
i) {1-Benzyl-5-[4-(trifluoromethyl)phenyl]-1H-indol-3-yl}(oxo)acetic acid;
j) {6-[4-(tert-Butyl)phenyl]-1-methyl-1H-indol-3-yl}(oxo)acetic acid;
k) [5-(4-Acetylphenyl)-1-benzyl-1H-indol-3-yl](oxo)acetic acid;
l) {1-Benzyl-5-[4-(trifluoromethoxy)phenyl]-1H-indol-3-yl}(oxo)acetic acid;
m) {1-Benzyl-4-[4-(trifluoromethyl)phenyl]-1H-indol-3-yl}(oxo)acetic acid;
n) {1-Benzyl-5-[4-(tert-butyl)phenyl]-1H-indol-3-yl}(oxo)acetic acid;
o) [1-Benzyl-5-(3-chloro-4-fluorophenyl)-1H-indol-3-yl](oxo)acetic acid;
p) {1-Benzyl-5-[3,5-bis(trifluoromethyl)phenyl]-1H-indol-3-yl}(oxo)acetic acid;
q) {1-Benzyl-7-[4-(trifluoromethoxy)phenyl]-1H-indol-3-yl}(oxo)acetic acid;
r) [1-Benzyl-7-(3-chloro-4-fluorophenyl)-1H-indol-3-yl](oxo)acetic acid;
s) {1-(4-tert-Butylbenzyl)-5-[4-(trifluoromethoxy)phenyl]-1H-indol-3-yl}(oxo)acetic acid;
t) {1-Benzyl-4-[4-(trifluoromethoxy)phenyl]-1H-indol-3-yl}(oxo)acetic acid;
u) [1-Benzyl-6-(3-chlorophenyl)-1H-indol-3-yl](oxo)acetic acid;
v) {1-Benzyl-5-[3-(trifluoromethoxy)phenyl]-1H-indol-3-yl}(oxo)acetic acid;
w) {1-(4-Methylbenzyl)-5-[4-(trifluoromethoxy)phenyl]-1H-indol-3-yl}(oxo)acetic acid;
x) {1-(4-Fluorobenzyl)-5-[4-(trifluoromethoxy)phenyl]-1H-indol-3-yl}(oxo)acetic acid;
y) [1-Butyl-5-(4-chlorophenyl)-1H-indol-3-yl](oxo)acetic acid;
z) [1-Butyl-5-(3-chlorophenyl)-1H-indol-3-yl](oxo)acetic acid;
aa) [1-Butyl-5-(3-methoxyphenyl)-1H-indol-3-yl](oxo)acetic acid;
bb) [1-Butyl-5-(4-methoxyphenyl)-1H-indol-3-yl](oxo)acetic acid;
cc) {1-Butyl-5-[4-(trifluoromethyl)phenyl]-1H-indol-3-yl}(oxo)acetic acid;
dd) [1-(4-tert-Butylbenzyl)-5-(3-methylphenyl)-1H-indol-3-yl](oxo)acetic acid;
ee) [1-(4-tert-Butylbenzyl)-5-(3-methoxyphenyl)-1H-indol-3-yl](oxo)acetic acid;
ff) [1-(4-tert-Butylbenzyl)-5-(4-tert-butylphenyl)-1H-indol-3-yl](oxo)acetic acid;
gg) [1-(4-tert-Butylbenzyl)-5-(3-chlorophenyl)-1H-indol-3-yl](oxo)acetic acid;
hh) [1-(4-tert-Butylbenzyl)-5-(4-chlorophenyl)-1H-indol-3-yl](oxo)acetic acid;
ii) [1-(4-tert-Butylbenzyl)-5-(2-methylphenyl)-1H-indol-3-yl](oxo)acetic acid;
jj) {1-(2-Ethylbutyl)-5-[4-(trifluoromethoxy)phenyl]-1H-indol-3-yl}(oxo)acetic acid;
kk) {2-[(Acetyloxy)methyl]-1-(4-methylbenzyl)-5-[4-(trifluoromethoxy)phenyl]-1H-indol-3-yl}(oxo)acetic acid;
ll) {2-(Hydroxymethyl)-1-(4-methylbenzyl)-5-[4-(trifluoromethoxy)phenyl]-1H-indol-3-yl}(oxo)acetic acid;
mm) {2-[(Acetyloxy)methyl]-1-benzyl-5-[4-(trifluoromethoxy)phenyl]-1H-indol-3-yl}(oxo)acetic acid;
nn) {1-Benzyl-2-(hydroxymethyl)-5-[4-(trifluoromethoxy)phenyl]-1H-indol-3-yl}(oxo)acetic acid;
oo) [5-(3-Chlorophenyl)-1-cyclopentyl-1H-indol-3-yl]-oxo-acetic acid;
pp) [5-(3-chlorophenyl)-1-(cyclobutylmethyl)-1H-indol-3-yl](oxo)acetic acid;
qq) [5-(3-chlorophenyl)-1-(3-methylcyclopropyl)-1H-indol-3-yl](oxo)acetic acid;
rr) [5-(3-chlorophenyl)-1-(cyclohexylmethyl)-1H-indol-3-yl](oxo)acetic acid;
ss) 5-(4-trifluoromethylphenyl)-1-(cyclopentyl)-1H-indol-3-yl](oxo)acetic acid;
tt) [5-(4-trifluoromethylphenyl)-1-(cyclobutylmethyl)-1H-indol-3-yl](oxo)acetic acid;
uu) [5-(4-trifluoromethylphenyl)-1-(3-methylcyclopentyl)-1H-indol-3-yl](oxo)acetic acid;
vv) [5-(4-trifluoromethylphenyl)-1-(cyclohexylmethyl)-1H-indol-3-yl](oxo)acetic acid;
ww) [5-(4-trifluoromethylphenyl)-1-(cyclopentylpropyl)-1H-indol-3-yl](oxo)acetic acid;
xx) [5-(3-trifluoromethylphenyl)-1-(cyclopentyl)-1H-indol-3-yl](oxo)acetic acid;
yy) [5-(3-trifluoromethylphenyl)-1-(cyclobutylmethyl)-1H-indol-3-yl](oxo)acetic acid;
zz) [5-(3-trifluoromethylphenyl)-1-(3-methylcyclopentyl)-1H-indol-3-yl](oxo)acetic acid;
aaa) [5-(3-trifluoromethylphenyl)-1-(cyclohexylmethyl)-1H-indol-3-yl](oxo)acetic acid;
bbb) [5-(3-trifluoromethylphenyl)-1-(cyclopentylpropyl)-1H-indol-3-yl](oxo)acetic acid; or
ccc) [5-(4-methoxyphenyl)-1-(cyclohexylmethyl)-1H-indol-3-yl](oxo)acetic acid;
or a pharmaceutically acceptable salt, solvate or ester thereof.

6. The composition of claim 1, wherein the compound is micronized [1-(4-tert-Butylbenzyl)-5-(3-methylphenyl)-1H-indol-3-yl](oxo)acetic acid, or a micronized pharmaceutically acceptable salt, solvate or ester thereof.

7. The composition of claim 6, wherein the composition comprises from 1% to 50% w/w of the compound.

8. The composition of claim 6, wherein the composition comprises from 3.33% to 33% w/w of the compound.

9. The composition of claim 6, wherein the composition comprises from 5% to 20% w/w of one or more surfactants.

10. The composition according to claim 6, wherein the composition comprises from 5% to 15% w/w of one or more surfactants.

11. The composition according to claim 6, wherein the composition comprises from 7% to 12.5% w/w of one or more surfactants.

12. The composition according to claim 6, wherein the composition comprises about 20% w/w of one or more surfactants.

13. The composition according to claim 6, wherein the composition comprises about 15% w/w of one or more surfactants.

14. The composition according to claim 6, wherein the composition comprises about 10% w/w of one or more surfactants.

15. The composition according to claim 6, wherein the composition comprises about 5% w/w of one or more surfactants.

16. The composition according to claim 9, wherein at least one of the surfactants of the invention is a sodium docusate, a cyclodextrin, a polysorbate, a centrimide, a d-alpha-tocopheryl polyethylene glycol, a sodium lauryl sulfate, or a poloxamer.

17. The composition according to claim 16, wherein at least one of the surfactants is d-alpha-tocopheryl polyethylene glycol 1000 succinate or sodium lauryl sulfate.

18. The composition according to claim 6, comprising from 1% to 35% w/w of one or more binders.

19. The composition according to claim 6, comprising from 3% to 30% w/w of one or more binders.

20. The composition according to claim 6, comprising from 5% to 25% w/w of one or more binders.

21. The composition according to claim 6, comprising from 10% to 20% w/w of one or more binders.

22. The composition according to claim 6, comprising about 15% w/w of one or more binders.

23. The composition according to claim 18, comprising at least one binder selected from the group consisting of microcrystalline cellulose, povidone, carbomers, polyoxamers, starch, methyl cellulose, chitosan, sodium alginate, sucrose, maltose, and gelatin.

24. The composition according to claim 6, comprising 1% to 15% w/w of one or more disintegrants.

25. The composition according to claim 6, comprising 2% to 12% w/w of one or more disintegrants.

26. The composition according to claim 6, comprising 2% to 10% w/w of one or more disintegrants.

27. The composition according to claim 6, comprising 2% to 8% w/w of one or more disintegrants.

28. The composition according to claim 6, comprising about 5% w/w of one or more disintegrants.

29. The composition according to claim 24, comprising at least one disintegrant selected from croscarmellose sodium, sodium starch glycolate, povidone and starch.

30. The composition according to claim 6, comprising from 0.01% to 5% w/w of one or more glidants.

31. The composition according to claim 6, comprising about 0.5% w/w of one or more glidants.

32. The composition according to claim 30, comprising glidants selected from colloidal silicon dioxide, talc, magnesium silicate or calcium silicate.

33. The composition according to claim 6, comprising from 0.01% to 5% w/w of one or more lubricants.

34. The composition according to claim 6, comprising about 0.5% w/w of one or more lubricants.

35. The composition according to claim 33, comprising lubricants selected from magnesium stearate, sodium stearyl fumarate or stearic acid.

36. The composition according to claim 6, comprising from 10% to 85% w/w of one or more fillers.

37. The composition according to claim 6, wherein the composition comprises from 1% to 50% w/w of the compound; and 5% to 20% w/w of one or more surfactants.

38. The composition according to claim 6, wherein the composition comprises from 1% to 50% w/w of the compound; 5% to 20% w/w of one or more surfactants; and from 1% to 35% w/w of binders.

39. The composition according to claim 38, further comprising from 1% to 15% w/w of one or more disintegrants.

40. The composition according to claim 39, further comprising from 0.01% to 5% w/w of one or more glidants; from 0.01% to 1% w/w of one or more lubricants; and from 50% to 80% w/w of one or more fillers.

41. The composition according to claim 6, wherein the composition comprises from 3.33% to 33% w/w of the compound; and from 5% to 15% w/w of one or more surfactants.

42. The composition according to claim 6, wherein the composition comprises from 3.33% to 33% w/w of a compound of formula (I); and from 5% to 15% w/w of one or more surfactants; and from 3% to 30% w/w of binders.

43. The composition according to claim 42, further comprising from 2% to 12% w/w of one or more disintegrants.

44. The composition according to claim 43, further comprising from 0.01% to 5% w/w of one or more glidants; from 0.01% to 1% w/w of one or more lubricants; and from 50% to 80% w/w of one or more fillers.

45. A method of treating Alzheimer's disease comprising the administration of a composition according to claim 1 to a mammal in need thereof.

46. A method of increasing or normalizing levels of plasmin concentration comprising the administration of a composition according to claim 1 to a mammal in need thereof.
